(19) United States
(12) Patent Application Publication
(10) Pub. No.: US 2004/0191170 A1
(43) Pub. Date: Sep. 30, 2004

(54) Animal model for enteric pathogens

(76) Inventors: James J. Mond, Silver Spring, MD (US); Frederick J. Cassels, Laurel, MD (US); John F. Kokai-Kun, Frederick, MD (US)

Correspondence Address: FINNEGAN, HENDERSON, FARABOW, GARRETT & DUNNER, LLP, 1300 I STREET, NW, WASHINGTON, DC, 20005, US

(21) Appl. No.: 10/473,735

(22) PCT Filed: Apr. 3, 2002
(86) PCT No.: PCT/US02/08234

Related U.S. Application Data
(60) Provisional application No. 60/280,736, filed on Apr. 3, 2001.

(51) Int. Cl. A61K 49/00

(52) U.S. Cl. 424/9.2

(57) ABSTRACT

The present invention provides a reliable, low cost animal model for evaluating infections caused by enteric pathogens, including diarrheagenic Escherichia coli, such as enterotoxigenic, enterohemorrhagic, Shiga-toxin producing, and enteropathogenic E.coli. The animal model can be used for vaccine development and drug screening, including the screening of compounds that impair or inhibit the binding of enteric pathogens to host cells or compounds that inhibit the effects of toxins produced by the enteric pathogen. FIG. (1) represents the detection of CFA/I expressing ETEC in intestines and feces of ETEC-infected cotton rats using colony blots.

### FIELD OF THE INVENTION

The present invention relates to an animal model for enteric pathogens, including diarrheagenic Escherichia coli, such as enterotoxigenic, enterohemorrhagic, Shiga-toxin producing, and enteropathogenic E coli. More particularly, the invention relates to methods of using the animal model for vaccine development and drug screening, including the screening of compounds that impair or inhibit the binding of enteric pathogens to host cells or compounds that inhibit the effects of a toxin produced by the enteric pathogen.

### BACKGROUND

Diarrhea represents a significant problem for inhabitants of both developed countries and developing countries. In the United States, there are up to 38 million cases of diarrhea per year, resulting in up to 425 deaths in children under the age of five years. Black, Vaccine 11(2) 100-06 (1993). This disease is very costly as it results in up to 3.7 million visits to the doctor and approximately 220,000 hospitalizations per year. Id. In addition, sporadic outbreaks of infectious diarrhea can cause significant mortality as well.

Travelers from developed countries face a high likelihood that they will contract diarrhea when they visit a developing country. Approximately 50 million people travel from developed countries to developing areas each year. Ansdell et al., Med. Clin. North Am. 83(4) 945-73 (1999). About half of these travelers will contract an infectious diarrhea either during their trip or shortly after their return. Weekly Epidemiological Record 13: 97-104 (1999). Of course, military troops who are sent to developing countries are also vulnerable to infectious diarrhea.

Although problematic in the United States, the incidence of diarrhea in developing countries is much higher and the consequences more severe. It has been estimated that there are 1.5 billion episodes of diarrhea per year among children under the age of five in developing countries, resulting in 4 million deaths. Pediatr. Infect. Dis. J. 9:345-355 (1990). Thus, diarrhea is associated with one-third of all deaths in children under the age of five in developing countries.

A major cause of diarrheal diseases is bacterial infection. Enteric bacteria, such as Escherichia coli (E. coli) commensally inhabit human intestinal tissue and are required for proper gut function. Although the commensal strains of E. coli are normally harmless and, indeed, necessary for optimal digestive function, several variant E. coli strains are virulent and cause diarrhea. These diarrheagenic strains have been categorized based on defined clinical symptoms and virulence mechanisms into at least seven groups. These groups include enteroinvasive (EIEC), enteropathogenic (EPEC), enterotoxigenic (ETEC), enterohemorrhagic (EHEC), diffusely adherent (DAEC) enteroaggregative (EAEC), and Shiga toxin-producing (STEC) E. coli. Diarrheagenic E. coli strains are able to colonize the intestinal mucosal surface despite peristalsis and competition with the normal flora of the gut. Unlike nonpathogenic E. coli, some diarrheagenic E. coli strains express fimbrial antigens that facilitate their ability to colonize the intestine and mediate adherence to the small or large bowel mucosa. Two of these diarrheagenic groups (i.e., ETEC and EHEC) present major challenges to human health. ETEC is associated with diarrheal disease in the developing world and is the predominant etiologic agent causing travelers diarrhea in adults from the developed world visiting areas where ETEC infection is endemic. Nataro et al., Clinical Microbiology Reviews 11(1) 142-201 (1998). ETEC is also the most frequently isolated pathogen from children under the age of five in these areas of the world. Weekly Epidemiological Record 13: 97-104 (1999). Although ETEC requires a high dose of bacterial exposure for symptomatic infection, large numbers of infectious ETEC bacteria are shed from the stool of affected individuals, thereby providing a reservoir of infectious bacteria in the endemic regions. ETEC strains produce the heat labile enterotoxin (LT) which is similar to the cholera enterotoxin and/or heat stable enterotoxin (ST). These toxins are factors that contribute to diarrhea. Therefore, finding agents that inhibit these toxins or the effects thereof may prove useful in reducing or inhibiting diarrhea.

EHEC are the causative agent of “hemorrhagic colitis” (HC) and the more serious sequelae, hemolytic uremic syndrome (HUS). Spika, J. et al., J. Pediatr., 109: 287-291(1986); Remis, R., Ann. Intem. Med., 101:624-626 (1984); Riley, L. et al., N. Engl. J. Med., 308:681-685 (1983). HC is characterized by severe abdominal pain, initially watery diarrhea followed by copious, bloody diarrhea, with little or no fever. HUS is one of the complications resulting from HC and is characterized by acute renal failure, thrombocytopenia, and microangiopathic hemolytic anemia. HC occurs most frequently in developed countries, and most outbreaks of this disease have been associated with the consumption of contaminated meats (e.g., undercooked ground beef) and dairy products (e.g., raw milk). Doyle et al., J. Appl. Environ. Microbiol. 53:2394 (1987); Samadpour et. al., J. Appl. Environ. Microbiol. 60:1038 (1994).

E. coli serotype O157:H7 is the most frequent EHEC isolate in the United States, but many other serotypes of E. coli that are capable of causing equally devastating food-borne outbreaks of HC and HUS have been identified. Presently, EHEC and, in particular, E. coli O157:H7, are among the most serious bacterial pathogens confronting the public health and food safety agencies. One estimate ranks the total annual costs of O157:H7 infection alone as the fourth most costly food-borne pathogen in the United States. Weekly Epidemiological Record 14: 105-112 (1999).

The major virulence factor and a defining characteristic of EHEC is the production of Shiga toxins (Stx). EHEC and Shigella dysenteriae both produce a family of closely related cytotoxins that collectively will be called “Shiga toxins” for the purpose of this application (for a review, see O'Brien and Holmes, Microbiol. Rev., 51:206-220 (1987)).

The ability of many diarrheagenic E. coli to adhere to host epithelial cells is regarded as a prerequisite for the initial colonization of host tissue. The adhesion of diarrheagenic E. coli to host cells is often mediated by surface fimbriae, or pili, which recognize specific receptors on the host cell surface and allow the bacteria to colonize the intestinal mucosa. With respect to infection, the most important type of pili associated with enterotoxigenic E. coli strains are called colonization factors (CF) or coli surface antigens (CS). The presence of CF on pathogenic strains of E. coli facilitate the attachment of the organism to intestinal receptor molecules in a species and tissue specific fashion. Cassels and Wolf, Indus. Microbiol. 15:214 (1995). In ETEC, there are currently two recognized CF families, the CFA/I family (including CFA/I, CS1, CS2, CS4, CS17, CS19, and PCF O166) and the CS5 family (including CS5, CS7, CS20, and PCF O20). Other CF have been described (e.g., CS3, CS6, PCF O148, PCF O159) but have no associated family members. The most common phenotypes are CFA/I, CFA/II, and CFA/IV, accounting for up to 75% of known, well characterized ETEC. McConell et al, Epidemial Infect, 106:477-484 (1991). Given important role of CFs in mediating adherence to and colonization of intestinal mucosa, purfied CFs, or fragments thereof, are commonly included in new vaccines. Weekly Epidemiological Record 13: 97-104, p. 98 (1999). Several potential oligosaccharide receptors have been identified for CF and include the asialo GM1 glycolipid structure (βGal(1-3)βGalNAc(1-4)βGal(1-4)βGlc-ceramide) as well as several sialic acid containing glycoconjugates, as described in U.S. Pat. No. 5,891,860 to Heerze et al. In addition, compounds that impair or inhibit CFs from binding to the appropriate receptors on host epithelial cells may be useful for treating or preventing pathogen infection. These compounds may act directly or indirectly on the CF, or they may act directly or indirectly on the corresponding host cell receptor. For example, Bromelain, a proteolytic abstract from pineapple stems, appears to inhibit ETEC attachment in pigs and rabbits by proteolytically modifying the receptor attachment sites in the intestinal mucosa. Mynott et al., Gut, 38(1):28-32, (1996); Mynott et al., Gastroenterology, 113(4):1425, (1997); and Chandler and Mynott, Gut, 43(2):196-202, (1998).

Other important causes of bacterial-induced diarrhea include the following enteric pathogens: Campylobacter jejuni, Shigella spp. (e.g., Shigella dysenteriae), Vibrio spp. (e.g., Vibrio cholerae), Salmonella spp and Clostridia spp (e.g., Clostridium difficile). Diarrhea may also be caused by enteropathogenic viruses such as rotavirus. Ansdell et al., Med. Clin. North Am. 83(4) 945-73 (1999). Similarly to diarrheagenic E. coli strains many of these other enteric pathogens have the ability to colonize the intestinal mucosa, where they can adhere to host epithelial cells or produce toxins. Farthing, M. J., Trans. R. Soc. Trop. Med. Hyg. 79:569-76 (1985).

The preferable treatment for pathogen-induced diarrhea is the prevention of colonization. While this can be accomplished through public health methods, implementation of an improved public health regime is difficult and uncertain in the developing world. In addition, despite the superior sanitary systems in the developed world, outbreaks of diarrhea are not uncommon. Therefore, the development of effective vaccines is an attractive approach for the prevention of pathogen-mediated diarrheal diseases. In fact, the World Health Organization has recently designated ETEC as a target enteric pathogen to be controlled by vaccination. Alves et al., Brazilian J. of Med. Bio. Res. 32: 223-229 (1999).

One major factor hampering the development of vaccines for enteric pathogens is the lack of an adequate, simple and low cost animal model in which to test vaccines. In order to design an appropriate human vaccine to combat enteric pathogens, a more practical animal model that closely resembles the pathophysiology of these infections in humans is needed. Animal models currently in use have significant limitations. For example, the ETEC animal model most commonly used, the reversible intestinal tie adult rabbit diarrhea model, requires physical ligation of a segment of the intestine and is traumatic for the animal. Moon et al., Ann. N.Y. Acad. Sci. 176:197-211 (1971). This model is extremely cumbersome, requiring abdominal surgery on each test animal. Pigs and other large farm animals can also be infected with some strains of ETEC. Smith et al., J. Pathol. Bacteriol. 93:499-529 (1967). However, due to the costs associated with maintaining large animals, this model is not practical for use in the laboratory.

Thus, there is an important need to develop a simple and low cost animal model to test the efficacy of candidate vaccines. Also needed is a convenient in vivo test procedure that can be used to evaluate potential therapeutic agents for efficacy against enteropathogenic infections, including diarrheagenic E. coli infections.

### SUMMARY OF THE INVENTION

The present invention addresses these needs by providing a reliable, low cost animal model for enteropathogenic infections (i.e, infections caused by enteric pathogens). This animal model is preferably used to evaluate the efficacy of vaccines and therapeutic agents against enteric bacterial infections, including those caused by E. coli, C. jejuni, Shigella spp., Vibrio spp., Salmonella spp. and Clostridia spp. Similarly, this animal model can also be used to evaluate enteric viral infections. In one embodiment, the model is used to evaluate diarrheagenic E. coli infections. Thus, an object of the present invention is to provide a method of using the animal model to develop vaccines and to evaluate the efficacy of therapeutic agents in the prevention and treatment of enteropathogenic infections, including diarrheagenic E. coli infections.

Since adherence to host epithelial cells can mediate the initial colonization of host tissue, animals used for vaccine testing should express receptors that allow these pathogens to bind and infect host cells. Although we do not wish to be limited by any theory or hypothesis, the present invention suggests that the intestines of adult animals may express lower levels of receptors for enteric pathogens, such as diarrheagenic E. coli, whereas cells in the intestines of neonates may express much higher levels of functional receptors. Alternatively, neonates may have fewer normal flora in their intestines, thus providing less competition for the enteric pathogen. This latter theory is consistent with our observation that treating older animals with a clearing agent to reduce normal intestinal flora, for example an antibiotic, such as ampicillin or streptomycin, alone or in combination, or acidified water, increases susceptibility to infection by the pathogen.

The animal model of this invention is particularly suitable for the development of vaccines against enteric pathogens, including diarrheagenic E. coli. In this respect, the present invention provides a method for evaluating the potential of an agent or a combination of agents to prevent an enteropathogenic infection or reduce a symptom associated with the infection, comprising: (a) administering the agent or combination of agents to a rodent, where prior to administering the agent or combination of agents the rodent is either (i) prematurely weaned, (ii) treated with one or more clearing agents, or (iii) prematurely weaned and treated with one or more clearing agents; (b) infecting the rodent with an enteric pathogen, which may be a diarrheagenic E. Coli strain, for example, a strain of ETEC, EHEC, STEC, or EPEC; and (c) evaluating the effectiveness of the agent or the combination of agents in the prevention of or reduction of symptoms associated with the enteropathogenic infection. Alternatively, the rodent can be treated with one or more clearing agents to reduce normal intestinal flora after administering the agent or combination of agents to the rodent.

In one embodiment, this method is used to evaluate antibody-mediated protection against infection by an enteric pathogen. The antibodies to be tested may be passively transferred to the test animal either prior to or concurrently with infection. Alternatively, the efficacy of antibody-mediated protection can be tested by inducing in female rodents an antibody response against at least one antigen from the enteric pathogen and evaluating the resistance of their offspring to infection by the enteric pathogen. This latter approach relies on the maternal transfer of antibodies before birth and during the suckling period. In addition, since we have demonstrated that older animals can be used in this model, antibody-mediated protection can be evaluated in adult animals by direct immunization without having to rely on passive or maternal transfer of antibodies. This method involves inducing an antibody response against at least one antigen from the enteric pathogen in the adult animal, treating the an animal with one or more clearing agents, infecting the animal with the enteric pathogen, and evaluating the resistance of the animal to infection by the enteric pathogen.

The invention also relates to a convenient in vivo test procedure that can be used to evaluate potential therapeutic agentsfor efficacy against enteropathogenic infections. These therapeutic agents include, but are not limited to, antibodies, antibiotics, compounds that inhibit a toxin produced by the pathogen or the effects of the toxin, and compounds that impair or inhibit pathogen attachment to host cells, including host cells in the intestinal mucosa. As one example, proteases, such as Bromelain, which proteolytically modify host receptors involved in pathogen colonization, can be tested in this system. In particular, this aspect of the invention involves a method for evaluating the potential of an agent or a combination of agents to treat an enteropathogenic infection, comprising: (a) infecting a rodent with an enteric pathogen, where prior to infection, the rodent is either (i) prematurely weaned, (ii) treated with one or more clearing agents to reduce normal intestinal flora, or (iii) prematurely weaned and treated with an agent to reduce normal intestinal flora; (b) administering the agent or combination of agents to the rodent prior to, concurrently with, and/or shortly after infection; and (c) evaluating the effectiveness of the agent or the combination of agents in preventing the spread of infection, reducing pathogen load, or reducing the severity or length of symptomology.

The accompanying drawings, which are incorporated in and constitute a part of this specification, illustrate several embodiments of the invention and together with description, serve to explain the principles of the invention.

### BRIEF DESCRIPTION OF THE DRAWINGS

FIG. 1 represents the detection of CFA/I expressing ETEC in intestines and feces of ETEC-infected cotton rats using colony blots.

FIG. 2 represents the detection of CS6 expressing ETEC in feces of ETEC-infected cotton rats.

It is to be understood that both the foregoing general description and the following detailed description are exemplary and explanatory only and are not restrictive of the invention, as claimed.

### DETAILED DESCRIPTION OF THE INVENTION

In accordance with the present invention, it has been found that infant rodents prematurely weaned from their mothers produce diarrhea following infection with an enteric pathogen and, therefore, provide a useful in vivo model for enteropathogenic infections, including diarrheagenic E. coli infections, such as those caused by ETEC, EHEC, STEC, and EPEC. Without limitation to any theory or hypothesis, the present invention suggests that infant rodents, particularly those that have been prematurely weaned from their mothers, express receptors that permit pathogenic enteric bacteria efficiently to bind to and infect host epithelial cells. Thus, premature weaning may stop or delay expression of receptors that are not conducive for colonization of diarrheagenic E. coli. Altematively, neonates may have fewer normal flora in their intestines, thus providing less competition for an enteric pathogen.

Since adherence to host epithelial cells can mediate the initial colonization of host tissue, animals used for vaccine testing should express receptors that allow these pathogens to bind and infect host cells. These receptors may be down regulated in adult rodents, the intestines of which express low levels of receptors for enteric pathogenic bacteria. Consequently, adult rodents may not develop disease-grade diarrhea following infection with enteric pathogens, such as diarrheagenic E. coli. It may be possible, however, under the right conditions, to induce diarrhea in older animals. For example, pretreating an animal with a clearing agent that reduces the normal intestinal flora may increase the susceptibility of the animal to infection by an enteric pathogen.

The animal model of the present invention can be advantageously used, for example, to develop vaccines or to evaluate potential therapeutic agents for efficacy against enteropathogenic infections. The effectiveness of an agent as a vaccine or a therapy, or alternatively the resistance of an animal to infection, can be evaluated by any means that directly or indirectly measures a symptom associated with an infection, such as the pathogen load following infection. For example, an agent's efficacy can be directly measured by determining bacterial load found in the intestine, or feces or free toxin in intestine or feces, when appropriate. Alternatively, the agent's efficacy can be evaluated indirectly by comparing quality and/or volume of diarrhea produced by rodents treated with the agent to that produced by non-treated control animals. Alternative parameters that can be used to evaluate the efficacy of an agent include mortality, morbidity, weight, or water consumption of the infected animal.

The observation that neonatal receptors may mediate the binding and infection of host epithelial cells by enteric pathogens, suggests that the animal model of this invention can be used with any infant animal that exhibits a similar expression pattern of neonatal receptors. On the other hand, if neonates provide a useful model for a different reason, for example, they have fewer normal flora, and therefore, present less competition for the invading pathogen, this model should be able to accommodate diverse expression patterns of neonatal receptors in infant rodents. In addition, adult animals may be used in this model if they have been treated with a clearing agent that reduces the normal flora making them more susceptible to pathogenic infection. The clearing agent may be administered in a single dose or in multiple doses given at different times.

The clearing agent can be any agent that reduces the normal flora of the rodent. For example the clearing agent can be an antibiotic, or combination of antibiotics, including but not limited to β-lactams, such as penicillin and ampicillin; aminoglycosides, such as streptomycin, kanamycin, amikacin, spectimomycin, gentamicin, tobramycin, and netilmicin; cephalosporins, chloramphenicol, erythromycin, vanomycin, tetracycline, and the like. Other antibiotics are described in Zinsser Microbiology 20^{th} Edition (W. Joklik, ed., 1992) Appleton & Lange Pub., East Norwalk, Conn., which is incorporated herein by reference in its entirety. Acidified water is another clearing agent. Any acid can be used to acidify the water to a pH about 1.5, 2, 3, or 3.5, or any range subsumed therein, including hydrochloric acid, sulfuric acid, nitric acid, citric acid, phosphoric, formic, acetic acid, carboxyl acid and the like. Buffered solutions having a pH of about 1.5, 2, 3, or 3.5, or any range subsumed therein, may also be applicable in the practice of the present invention.

Animals suitable for use in the practice of this invention include rodents and rodent-like animals such as mice, hamsters, rabbits, guinea pigs, ferrets, chinchilla, rats, and cotton rats. In one embodiment, the animals are infant or neonatal rodents of approximately 1-14 days old. As explained above, however, under the right conditions, diarrhea may also be induced in older animals.

As used in this application, the term “prematurely weaned” refers to animals that are weaned from their mothers at some time before the normal weaning time. For instance, in the cotton rat model, infants are normally weaned from their mothers at about day 14, i.e., 14 days after birth. Thus, as used in this application, cotton rats that have been prematurely weaned refer to animals that have been weaned from their mothers before day 14. When using prematurely weaned cotton rats in embodiments of this invention, the animals may be weaned from their mothers within 10 days of birth, and may be weaned within about seven days after birth.

The cotton rat, genus Sigmodon (including but not limited to S. alstoni, S. fulviventer, S. alleni, S. arizonae, S. hispidus, S. inopinatus, S. leucotis, S. mascotensis, S. ochrognathus, S. peruanus), is unique among small laboratory animals in its susceptibility to a wide variety of human infectious agents. Its first use in the study of human infection was reported in 1937, when its susceptibility to endemic (“scrub”) typhus was described. During World War II the cotton rat was used to prepare a vaccine against endemic typhus, which was given to British troops in Southeast Asia. In 1939 the cotton rat became the first non-primate model of paralytic poliomyelitis, and was the model of choice for polio for over a decade. In addition, its susceptibility to human respiratory syncytial virus (RSV) led to the discovery that high-titered antibody to RSV could prevent life-threatening pneumonia in high-risk infants.

The cotton rat's unique susceptibility to human adenoviruses has led to its use in pathogenesis studies, as well as gene therapy studies that employ human adenovirus as a delivery system for a therapeutic gene. In fact, use of the cotton rat in gene therapy has proven so reliable that the Food and Drug Administration now requires studies in cotton rats of certain forms of gene therapy prior to approval to test them in humans. Other human pathogens which have been studied successfully in cotton rats include parainfluenza virus (types 1, 2 and 3), influenza virus (types A and B), Venezuelan equine encephalitis, epidemic typhus, Mycoplasma pneumoniae, enteroviruses, tuberculosis, rickettsial spotted fever, Rift Valley Fever virus, vesicular stomatits virus and herpes simplex virus. It has also been reported that cotton rats can be infected with HIV-1. Langley et al., PNAS 95:14355-60 (1998). Thus, these studies demonstrate the value of the cotton rat as a reliable model of human infection.

Inbred cotton rats (e.g., Sigmodon hispidus and Sigmodon fulviventer) are currently produced by Virion Systems, Inc., Rockville, Md., for commercial sale. Virion Systems, Inc. is licensed by the United States Department of Agriculture to produce cotton rats for commercial sale. Breeding stock of the same species is also available from the National Center for Research Resources, Bethesda, Md., which is part of the National Institutes of Health.

### EXAMPLE 1

### Induction of Diarrhea in Prematurely Weaned 7-Day Old Cotton Rats

For each experiment, three or four sets of five infant cotton rats (Sigmodon hispidus) were used. The cotton rats were housed under standard conditions with five animals per cage. They were fed a diet of standard rat/mouse chow and water supplemented with fresh apples.

At seven days after birth, the infant cotton rats were removed from their mothers and prematurely weaned. On the day prior to the experiment, the seven-day old infant cotton rats were lavage-tube fed 300 μl of acidified water (HCI; pH 2) and food was withheld. Acidified water was provided ad libitum. On the day of the experiment, each cotton rat was lavage-tube fed a 300 μl aliquot of a filter-sterilized, 20% sucrose solution containing either 1) approximately 5×10⁷ to 5×10⁹ organisms of the ETEC strain H10407 (Evans et al., Infect Immun 12(3): 656-67 (1975)); 2) approximately 5×10⁷-5×10⁹ organisms of the ETEC strain B7A (DuPont et al., New Eng J Med 285: 1-9 (1971); 3) approximately 5×10⁷ to 5×10⁹ organisms of HS, a non-toxigenic, human commensal E. coli strain (DuPont et al., New Eng J Med 285: 1-9(1971)); or 4) no E. coli as a control. The lavage tube was a soft polypropylene catheter with a 24-gauge indwelling that was inserted through the esophagus into the stomach to facilitate ingestion of the bacteria. For the infant cotton rats in groups 1), 2), and 3), the E. coli was maintained in the drinking water (20% sucrose solution) overnight following infection at a concentration equal to 1/10 of the infection dose.

In all experiments, at least 80% of the ETEC-infected animals developed persistent diarrhea of grade 2 or higher within 24 hours after infection. Diarrhea generally abated in these animals by 7 days but may last longer. Diarrhea has been detected as long as 14 days post inoculation. The infant cotton rats treated with either the human commensal E. coli strain or no E. coli and kept under the same housing conditions as the ETEC-infected cotton rats, did not develop diarrhea. The results of these experiments are summarized in Table 1. Both strains used in this study (i.e., H10407 and B7A) are human clinical isolates.

Diarrhea was graded on a score of 1-6, using the following scale: 0=no stool; 1=normal stool; 2=normal stool with free water; 3=loose stool; 4=gel-like stool; 5=liquid stool; 6=dead.

TABLE 1
Incidence of Diarrhea (Grade 2 or higher)

| | Experiment 1 | Experiment 2 | Experiment 3 |
|---|---|---|---|
| H10407 strain | 5/5 | 4/5 | 4/4 |
| B7A strain | 4/4 | 5/5 | 5/5 |
| non-diarrheagenic | 0/5 | 0/5 | 0/6 |
| E. coli strain HS | | | |
| Control (no infection) | 0/5 | | |

### EXAMPLE 2

### Challenge of Normally Weaned 14-Day Old Cotton Rats with ETEC

The experiment described in Example 1 was repeated using 14-day old cotton rats that had not been prematurely weaned from their mothers. When these 14-day old cotton rats were used, only 1 of 8 animals infected with ETEC developed diarrhea. These results, in combination with the results of the experiment described in Example 1, indicate that prematurely weaning the infant cotton rats from their mothers may regulate the expression of receptors that permit these pathogenic enteric bacteria to bind and infect host cells. Alternatively, these results may reflect a normal pattern of receptor down-regulation occurring in this period, or the younger cotton rats may have fewer normal flora in their intestine, thus providing less competition for the enteric pathogen.

### EXAMPLE 3

### Induction of Diarrhea in Prematurely Weaned 14-Day Old Cotton Rats

In an attempt to induce diarrhea in older cotton rats, 20 cotton rats were weaned at seven days as in Example 1. These rats were divided into four groups of five. Each group was treated differently prior to challenge with ETEC H10407 at day 14. Group 1 received 300 μl of acidified water by lavage tube 24 hours prior to bacterial challenge. Group 2 received 300 μl acidified water by lavage tube at 4 days and at 24 hours prior to bacterial challenge and were maintained on acidified water ad libitum throughout the four-day period. Group 3 received 300 μl of acidified water by lavage tube at seven days, four days and 24 hours prior to bacterial challenge and were maintained on acidified water ad libitum throughout the seven-day period. Group 4 received 1.5 mg of streptomycin in water (5 g/1) by lavage tube at four days prior to bacterial challenge and 300 μl of acidified water 24 hours prior to bacterial challenge. Group 4 animals were maintained on acidified water ad libitum throughout the four-day period prior to challenge. On the day of bacterial challenge, each animal received about 4.2×10⁹ H10407 in 300 μl 20% sucrose in water and a 1/10 dilution of H10407 in 20% sucrose ad libitum overnight. Animals were monitored for diarrhea as described above.

TABLE 2
Incidence or diarrhea (grade 2 or higher)
in 14-day old cotton rats

| | | Animals with |
|---|---|---|
| Group | Treatment | diarrhea |
| 1 | single acidified water treatment | 0/5 |
| 2 | two acidified water treatments over 4 days | 2/5 |
| 3 | three acidified water treatments over 7 days | 3/5 |
| 4 | streptomycin treatment followed by acidified | 3/3¹ |
| | water over 4 days | |

¹Note:
two animals died in Group 4, unrelated to bacterial challenge or pretreatment.

### EXAMPLE 4

### Induction of Diarrhea in Prematurely Weaned 7-Week Old Cotton Rats

To further examine the induction of diarrhea in older animals, 7-week old cotton rats, pretreated with ampicillin and streptomycin, were challenged with bacteria. In this experiment, animals were weaned at seven days and kept until seven weeks old. Four days prior to bacterial challenge, animals were treated with 500 μl of ampicillin/streptomycin-containing water (5 g/l) by lavage tube. Animals were maintained on ampicillin/streptomycin-containing water (5 g/l) ad libitum until the day before bacterial challenge. On the day prior to challenge with ETEC H10407, animals were given 500 μl acidified water by lavage tube and maintained on acidified water ad libitum overnight. Animals were also fasted overnight. On the day of bacterial challenge, each animal was lavage-tube fed 500 μl of 20% sucrose containing about 2.5×10¹⁰ bacteria. The animals received a 1/10 dilution of H10407 in 20% sucrose ad libitum overnight. Three out of three cotton rats treated in this manner contracted diarrhea of grade 2 or higher by day 1.

### EXAMPLE 5

### Challenge of Normally Weaned 5-Week Old Cotton Rats with ETEC

In this experiment, cotton rats were normally weaned at day 14. Prior to bacterial challenge at 5 weeks, the animals were pretreated with ampicillin and streptomycin. Four days prior to bacterial challenge, animals were treated with 500 μl of ampicillin/streptomycin (5 g/1) in a 20% sucrose solution by lavage tube. Animals were maintained on ampicillin/streptomycin (5 g/1) in 20% sucrose ad libitum until the day before bacterial challenge. On the day prior to challenge, animals were given 500 μl acidified water by lavage tube and maintained on acidified water ad libitum overnight. Animals were also fasted overnight. On the day of bacterial challenge, each animal was lavage-tube fed 500 μl of 20% sucrose containing 1) about 4×10¹⁰ ETEC H10407, 2) about 4×10¹⁰ of a commensal HS strain, or 3) no bacteria. Additionally, the animals received a 1/10 dilution of bacteria in 20% sucrose ad libitum overnight. On day three following challenge, 4 out of 4 animals in the ETEC group had diarrhea, while 1 out of 4 in the no bacteria group, and 1 out of 2 in the HS group had diarrhea.

The results of Examples 3-5 suggest that more stringent treatments to eliminate competition in the intestine (e.g., longer acidified water treatment, streptomycin treatment, or streptomycin/ampicillin treatment) permit colonization by ETEC and thus induction of diarrhea in adult animals. These results also suggest that diarrhea can be induced in even older animals with increased manipulations.

### EXAMPLE 6

### Detection of ETEC in Infected Cotton Rats

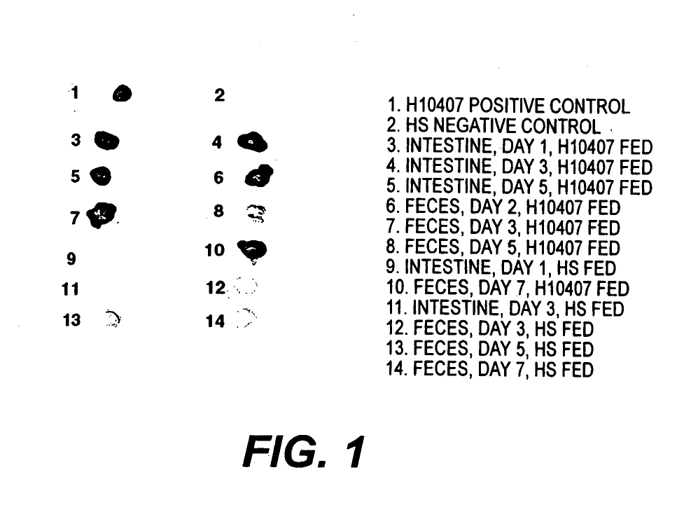

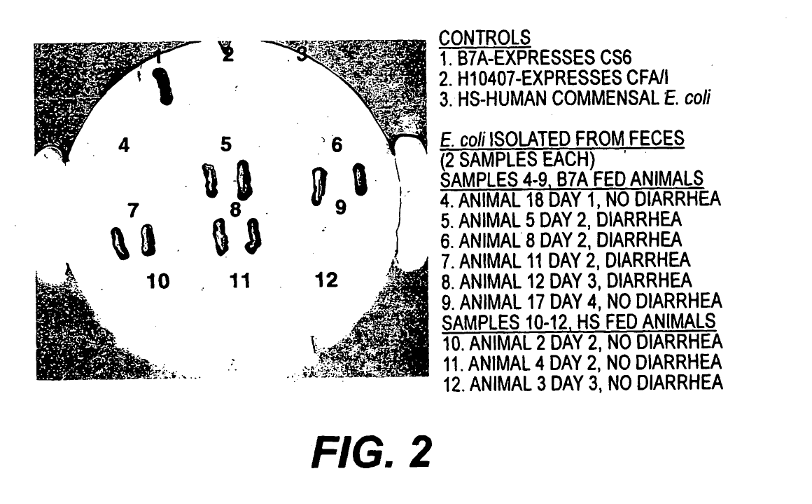

Cotton rats were infected as described in Example 1. At various days following infection, the infected animals were sacrificed and their small intestines were dissected and fecal matter was collected. The dissected small intestine or fecal matter was suspended in sterile saline and vortexed vigorously. E. coli released into the saline was isolated on MacConkey Agar. Colonies from MacConkey Agar were transferred to colonization factor antigen (“CFA”) agar to induce production of ETEC colonization factors. Following overnight growth, a sterile filter membrane was added to the CFA agar plate and the colonies were allowed to grow for two more hours. The membrane was then washed with Tris-buffered saline containing 0.5% Tween 20 to remove unbound agar and bacteria and then blocked using 5% non-fat dried milk. The membrane was then incubated for two hours in rabbit anti-CFA polyclonal serum at a 1:500 dilution or an anti-CS6 monoclonal antibody at 1:1000 dilution in 5% non-fat dried milk followed by one hour in horseradish peroxidase conjugated goat anti-rabbit or rabbit anti-mouse serum (1:500). The rabbit anti-CFA polyclonal serum recognizes CFA antigens, including the CFA/I antigen, which is expressed by the ETEC strain H10407. The anti-CS6 antibody recognizes the CS6 antigen, which is expressed by the ETEC strain B7A. Colony blots were developed with a specific peroxidase substrate. FIG. 1 demonstrates the presence of ETEC in the intestine and feces of ETEC strain H10407 infected cotton rats but not in rats infected with the human commensal E. coli strain HS. FIG. 2 demonstrates the presence of the CS6 expressing ETEC B7A strain in feces from animals with diarrhea. As shown in the controls, the anti-CS6 antibody specifically recognizes the CS6 expressing ETEC strain B7A and does not recognize the ETEC strain H10407 (which expresses CFA/I) or the human commensal E. coli strain HS. CS6 expressing E. coli was detected on days 2 and 3 in the diarrhea of animals challenged with B7A. CS6 expressing E. coli was not detected in the feces of H10407- or HS-challenged animals or in the feces of B7A-challenged animals showing no diarrhea on day 1.

### EXAMPLE 7

### Immunization of Cotton Rats with Anti-ETEC Antibodies

The ability of antibodies to passively transfer protective immunity can be investigated using the cotton rat model described in Example 1. It has been shown that ETEC-induced diarrhea stimulates the production of anti-ETEC antibodies and that this response is protective. Rudin et al., Epidemol Infect., 119:391-393 (1997). This demonstrates that humoral antibodies can mediate protection for ETEC-induced diarrhea.

Therefore, to test the efficacy of anti-ETEC antibodies, anti-ETEC antibodies (e.g., purified rabbit IgG or IgA anti-CFA/I antibody in saline) are administered (e.g., orally, i.v., or i.p.) at various doses (e.g., about 5, 10, 40, or 80 mg/kg or any range subsumed therein), prior to infection (e.g., 18-24 prior to infection) and/or concurrent with infection.

Systemic delivery of antibodies by way of the gastrointestinal tract may be achieved by any known systemic delivery system, including, by way of example, microsphere or nanosphere encapsulation. Microsphere sized poly(lactic-co-glycolic acid) (PLGA) capsules with an antibody core can be synthesized by forming a water-oil-water emulsion followed by solvent evaporation (McGinity and O'Donnell, Adv. Drug Deliv. Rev. 28(1):25-42 (1997)) or by a cryogenic process (Jones et al. Adv. Drug Deliv. Rev. 28(1):71-84 (1997)). Coating of the microspheres with chitosan, the partially deacetylated form of the polysaccharide chitin, may enhance mucoadhesion in the intestine and enhance the efficiency of delivery. Kawashima et al., Pharm. Dev. Technol. 5(1):77-85 (2000). Transmucosal transport and release may be achieved by synthesizing solid chitosan nanoparticles with antibodies distributed evenly within the polymer matrix. Since PLGA and chitosan are biodegradable and mediate a sustained release of the antibody, they reduce the need for repeated administrations of antibody. Altematively, microcapsules can be synthesized using an aqueous-based, enteric coating system. Litwin et al., Annals Allergy Asthma and Immunology, 77: 132-138, (1996); Litwin et al., J. Allergy Clin. Immunol., 100:30-38, (1997); Van Deusen et al., Annals Allergy Asthma and Immunology, 78:573-580, (1997); and Adachi et al., J. Travel Med., 7(6):304-308 (2000).

A high titer affinity purified rabbit anti-CFA/I antibody can be used as a positive control, since the available data demonstrate that this antibody is protective in humans. Freedman et al., J. Infect. Dis. 177: p. 662 (1998). Saline may also be administered as a control. Infected animals are observed for (i) any delay in onset of diarrhea, (ii) any alteration in severity of diarrhea, (iii) the duration of diarrhea, or lessening of symptoms (e.g., weight loss, ruffling of fur, level of physical activity).

Alternatively, the efficacy of anti-ETEC antibodies can be investigated by immunizing female cotton rats with an ETEC antigen (e.g., CFA/I in an adjuvant, such as complete Freund's adjuvant), at a dose intended to induce high titer anti-CFA/I antibody. The anti-CFA/I antibody titer can be measured at any time following immunization, preferably after 21-28 days. A high titer antibody is one that demonstrates a positive reaction in an ELISA reaction when diluted to 1:10,000 or greater. These animals may optionally receive one or more boost injections at any time following the initial inoculation, and preferably on or around day 7-14. When the cotton rats have high titer antibody, they are mated and their offspring are prematurely weaned and studied at about 7 days after birth for susceptibility to ETEC-induced diarrhea. In addition, older offspring may be studied, provided they are treated with an agent to reduce normal intestinal flora. This approach relies on maternal transfer of antibody to the fetus and provides an alternative system for evaluating the efficacy of antibody-mediated protection against infection by enteric pathogens.

### EXAMPLE 8

### Protection of Prematurely Weaned Seven-Day Old Cotton Rats From ETEC-Induced Diarrhea Using an Anti-CS6 Monoclonal Antibody

In this experiment, approximately 3×10⁹ B7A (expresses the CS6 antigen) or H10407 (expresses the CFA/I antigen) were preincubated with or without 1 mg of anti-CS6 monoclonal antibody (MAb) in 20% sucrose for 45 minutes. Groups of five seven-day old animals, treated as described in Example 1, were fed approximately 3×10⁹/animal B7A or H10407 with or without the anti-CS6 MAb. Animals were monitored for diarrhea, and the results are presented in Table 3.

TABLE 3
Incidence and grade of diarrhea

| | | | | |
|---|---|---|---|---|
| Animal | Day 1 | Day 2 | Day 3 | Day 6 |

Grp 1 B7A

| | | | | |
|---|---|---|---|---|
| 1 | 0 | 0 | 5 | 5 |
| 2 | 0(CB¹) | 5 | 5* | 6 (CB) |
| 3 | 0 | 0(CB) | 5 | 5 |
| 4 | 0 | 0(CB) | 5 | 5 |
| 5 | 0 | 0(CB) | 3 | 6 (CB) |

Grp 2 B7A + anti-CS6 MAb

| | | | | |
|---|---|---|---|---|
| 1 | 0 | 0 | 2 | 5 |
| 2 | 4 | 3 | 0 | 1 |
| 3 | 0 | 0 | 0 | 2 |
| 4 | 0 | 0 | 0 | 0 |
| 5 | 0(CB) | 0 | 0(CB) | 3 |

Grp 3 H10407

| | | | | |
|---|---|---|---|---|
| 1 | 0(CB) | 5 | 4 | 4 |
| 2 | 4 | 0(CB) | 3 | 5 |
| 3 | 0 | 0 | 0 | 0 |
| 4 | 3 | 3 | 0 | 0 |

Grp 4 H10407 + anti CS6 MAb

| | | | | |
|---|---|---|---|---|
| 1 | 0 | 0 | 2 | 0 |
| 2 | 3 | 5 | 5 | 5 |
| 3 | 0 | 0(CB) | 5 | 5 |

(*anus so raw it was bleeding)
¹CB indicates no diarrhea actually visible, but the fur around the anus is clearly matted with dried fecal material from diarrhea.
²Note:
Unrelated to bacterial challenge or pretreatment, one animal in Group 3 died and two animals in Group 4 died.

In this example, anti-CS6 MAb markedly reduced the number of cases of diarrhea and the severity of those cases in B7A fed animals while having little affect on the diarrhea of animals fed H10407. This example shows that the cotton rat is a useful model for evaluating potential interventions for bacterial diarrheal diseases.

### EXAMPLE 9

### Induction of Diarrhea by Enterohemorrhagic E. coli

To demonstrate that the cotton rat is a good and relevant model system for other enteric pathogens, diarrhea induced by enterohemorrhagic E. coli was examined. Seven-day old cotton rats were treated as described in Example 1. Groups of five animals were either challenged with E. coli strain 86-24 (EHEC 0157:H7), 87-23 (a Shiga toxin negative derivative of 86-24), delta 10 (an intimin negative derivative of 86-24) or the human commensal E. coli strain HS. The dose of bacteria was approximately 5×10⁷ bacteria in 300 μl 20% sucrose. Table 4 presents the results of this experiment.

TABLE 4
Incidence of Diarrhea (Grade 2 or higher)

| Bacterial challenge | Number of animals with diarrhea |
|---|---|
| 86-24 | 5/5 |
| 87-23 | 1/5 |
| delta 10 | 3/5 |
| HS | 0/5 |

This example demonstrates that cotton rats are also susceptible to diarrhea caused by other diarrheagenic E. coli.

### EXAMPLE 10

### Induction of Diarrhea by Enteropathogenic E. coli

To further demonstrate that the cotton rat is a good and relevant model system for other enteric pathogens, diarrhea induced by enteropathogenic E. coli was examined. Seven-day old cotton rats were treated as described in Example 1. A group of four animals was challenged with E. coli strain E2348/69 (EPEC 0127:H6) (Jerse et al., PNAS U.S.A. 87:7839-43 (1990)), and a group of five animals was challenged with the human commensal E. coli strain HS. The dose of bacteria was approximately 5×10⁷ bacteria in 300 μl 20% sucrose. Table 5 presents the results of this experiment.

TABLE 5
Incidence of Diarrhea

| Bacterial challenge | Number of animals with diarrhea |
|---|---|
| E2348/69 | 4/4 |
| HS | 0/5 |

Three animals developed diarrhea on day one and maintained it through day six. The fourth animal developed diarrhea between days three and six. By day six, each of the animals challenged with E2348/69 had grade 5 diarrhea. The control animals (receiving the HS strain) did not develop diarrhea. This example further demonstrates that cotton rats are susceptible to diarrhea caused by, other diarrheagenic E. coli.

The specification is most thoroughly understood in light of the teachings of,the references cited within the specification, all of which are hereby incorporated by reference in their entirety. The embodiments within the 'specification provide an illustration of embodiments of the invention and should not be construed to limit the scope of the invention. The skilled artisan recognizes that many other embodiments are encompassed by the claimed invention and that it is intended that the specification and examples be considered as exemplary only, with a true scope and spirit of the invention being indicated by the following claims.

What is claimed is:

1. A method for evaluating the potential of an agent or a combination of agents to treat an enteropathogenic infection, comprising:
(a) infecting a cotton rat with an enteric pathogen, wherein prior to infection, the cotton rat is either (i) prematurely weaned, (ii) treated with one or more clearing agents or (iii) prematurely weaned and treated with one or more clearing agents;
(b) administering the agent or combination of agents to the cotton rat; and
(c) evaluating the effectiveness of the agent or the combination of agents in preventing the spread of infection, reducing pathogen load, or reducing a symptom associated with infection.

2. The method of claim 1, wherein the enteric pathogen is a diarrheagenic E. coli strain.

3. The method of claim 2, wherein the diarrheagenic E. coli strain is ETEC, EHEC, STEC, or EPEC.

4. The method of claim 3, wherein the diarrheagenic E. coli strain is ETEC.

5. The method of claim 1, wherein prior to infection, the cotton rat is treated with one or more clearing agents.

6. The method of claim 5, wherein the one or more clearing agents is acidified water, at least one antibiotic, or acidified water and at least one antibiotic.

7. The method of claim 1, wherein prior to infection, the cotton rat is prematurely weaned and treated with one or more clearing agents.

8. A method for evaluating the potential of an agent or a combination of agents to prevent an enteropathogenic infection or to reduce a symptom associated with the infection, comprising:
(a) administering the agent or combination of agents to a cotton rat, wherein prior to administering the agent or combination of agents, the cotton rat is either (i) prematurely weaned, (ii) treated with one or more clearing agents, or (iii) prematurely weaned and treated with one or more clearing agents;
(b) infecting the cotton rat with an enteric pathogen; and
(c) evaluating the effectiveness of the agent or the combination of agents in preventing or reducing the symptom associated with infection.

9. The method of claim 8, wherein the enteric pathogen is a diarrheagenic E. coli strain.

10. The method of claim 9, wherein the diarrheagenic E. coli strain is ETEC, EHEC, STEC, or EPEC.

11. The method of claim 10, wherein the diarrheagenic E. coli strain is ETEC.

12. The method of claim 9, wherein the effectiveness of the agent or the combination of agents in preventing or reducing the symptoms associated with the infection is evaluated by measuring diarrhea produced by the cotton rat or weight loss.

13. The method of claim 10, wherein the agent is an antibody.

14. The method of claim 13, wherein the antibody is administered prior to or concurrent with infection by the diarrheagenic E. coli.

15. The method of claim 13, wherein the antibody is an anti-ETEC antibody.

16. The method of claim 8, wherein prior to infection, the cotton rat is treated with one or more clearing agents.

17. The method of claim 16, wherein the one or more clearing agents is acidified water, at least one antibiotic, or acidified water and at least one antibiotic.

18. The method of claim 8, wherein prior to infection, the cotton rat is prematurely weaned and treated with one or more clearing agents.

19. A method for evaluating the efficacy of antibody-mediated protection against an enteropathogenic infection, comprising:
(a) inducing in a cotton rat, an antibody response against at least one antigen from an enteric pathogen;
(b) treating the cotton rat with one or more clearing agents;
(c) infecting the cotton rat with the enteric pathogen; and
(d) evaluating the resistance of the cotton rat to infection by the enteric pathogen.

20. The method of claim 19, wherein the enteric pathogen is a diarrheagenic E. coli strain.

21. The method of claim 20, wherein the diarrheagenic E. coli strain is ETEC, EHEC, STEC, or EPEC.

22. The method of claim 21, wherein the diarrheagenic E. coli strain is ETEC.

23. The method of claim 20, wherein the one or more clearing agents is acidified water, at least one antibiotic, or acidified water and at least one antibiotic.

24. A method for evaluating the potential of an agent or a combination of agents to prevent an enteropathogenic infection or to reduce a symptom associated with the infection, comprising:
(a) administering the agent or combination of agents to a cotton rat;
(b) treating the cotton rat with one or more clearing agents;
(c) infecting the cotton rat with an enteric pathogen; and
(d) evaluating the effectiveness of the agent or the combination of agents in preventing or reducing the symptom associated with infection.

25. The method of claim 24, wherein the enteric pathogen is a diarrheagenic E. coli strain.

26. The method of claim 25, wherein the diarrheagenic E. coli strain is ETEC, EHEC, STEC, or EPEC.

27. A method for evaluating the efficacy of antibody-mediated protection against an enteropathogenic infection, comprising:
(a) inducing in a cotton rat, an antibody response against at least one antigen from an enteric pathogen;
(b) mating the female cotton rat with a male cotton rat;
(c) infecting the offspring of the female cotton rat with the enteric pathogen; and
(d) evaluating the resistance of the offspring to infection by the enteric pathogen.

28. The method of claim 27, wherein the offspring has been prematurely weaned prior to infection.

29. The method of claim 27, wherein the enteric pathogen is a diarrheagenic E. coli strain.

30. The method of claim 29, wherein the diarrheagenic E. coli strain is ETEC.

31. The method of any one of claims 1, 8, or 24, wherein the agent is a compound that impairs or inhibits the binding of the enteric pathogen to host cells.

32. The method of any one of claims 1, 8, or 24, wherein the agent is a compound that inhibits a toxin produced by the pathogen or an effect of the toxin.
